(19) United States
(12) Patent Application Publication
(10) Pub. No.: US 2016/0239988 A1
(43) Pub. Date: Aug. 18, 2016

(54) EVALUATION OF A DYNAMIC CONTRAST MEDIUM DISTRIBUTION

(71) Applicant: Miriam Keil, Erlangen-Dechsendorf (DE)

(72) Inventor: Miriam Keil, Erlangen-Dechsendorf (DE)

(21) Appl. No.: 15/011,810

(22) Filed: Feb. 1, 2016

(30) Foreign Application Priority Data
Feb. 12, 2015 (DE) 102015202494.6

(51) Int. Cl. G06T 11/00; G06K 9/46; A61B 5/055; G06T 7/00

(52) U.S. Cl. CPC G06T 11/008; G06T 7/0012; G06K 9/46; A61B 5/055; G06K 2009/4666; G06T 2207/10088

(57) ABSTRACT

The embodiments relate to a method for evaluation of a dynamic contrast medium distribution as well as to a medical imaging device and a computer program product for carrying out the method. The method includes providing measured values, establishing first reconstructed data from the measured values, generating reconstruction parameters from the first reconstructed data, and establishing second reconstructed data from the measured values using the generated reconstruction parameters.

This application claims the benefit of DE 10 2015 202 494.6, filed on Feb. 12, 2015, which is hereby incorporated by reference in its entirety.

### TECHNICAL FIELD

The embodiments relate to a method for evaluation of a dynamic contrast medium distribution as well as to a medical imaging device and a computer program product for carrying out the method.

### BACKGROUND

Dynamic contrast medium examinations are used by radiologists for detection and characterization of illnesses. Diagnoses such as the diagnosis of lesions, (in the liver or in the prostrate, for example), by dynamic magnetic resonance tomography (MRT) may be refined in this way. Here, a patient is injected with a contrast medium and, during this procedure or subsequently, time-dependent MRT measurements are carried out. An arterial phase, a venous phase, and/or a late phase may be observed. Here, the measurement period may be correctly selected relative to the time of the contrast medium injection. This timing is important for optimum detection of the arterial and venous phase and to trace the accumulation or distribution of the contrast medium. Contrast medium accumulates in different lesions at different times, which is why it is important not to measure just one phase, in order to insure that all lesions are detected.

### SUMMARY AND DESCRIPTION

The scope of the present invention is defined solely by the appended claims and is not affected to any degree by the statements within this summary. The present embodiments may obviate one or more of the drawbacks or limitations in the related art.

A method is provided herein that makes it possible to advantageously evaluate a dynamic contrast medium distribution, and also a medical imaging device and a computer program product for carrying out the method.

Accordingly, the method for evaluating a dynamic contrast medium examination includes the following acts: providing measured values; establishing a first reconstructed data from a first subset of the measured values; generating reconstruction parameters from the first reconstructed data; and establishing second reconstructed data from a second subset of the measured values using the generated reconstruction parameters.

This method enables the establishment of the second reconstructed data to be carried out in a more targeted manner, since additional information is included here, namely specific reconstruction parameters, which are generated on the basis of the first reconstructed data. Thus, the second reconstructed data may be restricted, for example, so that this data only defines a specific, (e.g., temporal), range. This might possibly allow important regions to be separated from unimportant regions, so that the establishment of the second reconstructed data is only carried out within relevant regions of interest.

The measured values may be raw data and/or data that has been recorded by a device, (e.g., a magnetic resonance device), within an acquisition time. Further processing of this raw data and/or data may possibly have been dispensed with. Thus, these measured values may be data that is written directly and/or unprocessed into a memory during recording. Furthermore, however, it is conceivable for the measurement data to have already been further processed before being provided.

The measured values may be provided as a part of carrying out a measurement. The measured values may also be provided by accessing stored measurement data that was recorded at an earlier point in time. These measured values may be dynamic or time-dependent, e.g., they may map a dynamic process, such as the progress of a contrast medium, which has a temporal development and/or a movement for example.

The first reconstructed data may represent a four-dimensional (4D) dataset including a time dimension as well as three space dimensions. For example, the 4D dataset may include at least one 5-tuple, which includes a space coordinate consisting of three values, a time value and an amplitude value.

The establishment of the first reconstructed data may be applied to all measured values, e.g., without restriction in respect of the time values of the dynamic measured values. Thus, the selection of a time range may be dispensed with, through which the method may be simplified. The first reconstructed data may extend over the entire time range of the dynamic measurement.

The first reconstructed data may be established using standard parameters, e.g., parameters that are not created specifically for an individual measurement. It is also conceivable for one standard parameter set to be selected from different standard parameter sets.

The first and the second subset of the measured values may include all or a part of the measured values provided. Furthermore, the first and the second subset of the measured values may be the same or different. In particular, the second subset may be a subset of the first subset.

One form of embodiment makes provision for the measured values to include imaging information of an examination, e.g., of a living human or animal being. Through the measured values, e.g., measured magnetic resonance signals, images of the examination object or of a part thereof, (e.g., vessels and/or internal organs and/or other parts of a human or animal body), may be generated. In addition a contrast medium, e.g., a substance containing Gadolinium, which is located in the examination object, may be detected. This enables a distribution of the contrast medium in a patient's body to be investigated as a function of time and/or for a bolus course to be traced.

Advantageously, the acquisition time of the measured values provided includes the point in time or the period of the accumulation of a contrast medium at the examination object. This enables it to be insured that the entire bolus course of the dynamic contrast medium examination may be evaluated.

It is further proposed that the measured values are detected by a medical imaging device. Conceivable modalities include Magnetic Resonance Tomography (MRT), Computed Tomography (CT), and sonography. These imaging methods are well suited for presenting structures and functions of a body using a contrast medium.

In MRT measurements the detected measurement data may be detected using a sparse matrix technique, e.g., a GRASP (Golden-Angle Radial Sparse Parallel), technique. In the sparse matrix technique, during the detection of the measured values, a data matrix, (e.g., a k-space matrix), is only partly filled (sparse). Through this compressed sensing of the k-space a rapid dynamic data detection is made possible, so that the sparse matrix technique is suitable for dynamic contrast media examinations.

In one embodiment, the first reconstructed data has a smaller, (e.g., temporal or spatial), resolution than the second reconstructed data. This enables the volume of data to be processed for establishment of the first reconstructed data to be reduced, so that the reconstruction time may be shortened and/or any requirements on a processing unit for carrying out the reconstruction may be reduced.

In a further form of embodiment, at least one Region Of Interest (ROI) is defined for generation of reconstruction parameters. The region of interest may include a volume and/or a surface within the examination object. For example, it is conceivable that a point in time may be selected from the first reconstructed data, which may include a 4D dataset and thus time values, and that image data assigned to this selected point in time is provided, for example, as a 3D dataset. Then, at least one region of interest may be determined on this dataset.

The selected point in time is advantageously selected so that the image data assigned to this selected point in time is informative, in order to enable the definition of the at least one region of interest to be carried out in a targeted manner. The selected point in time may be preset or selected individually for a specific measurement. In one variant, the selected point in time corresponds to a Care-Bolus time, which may be understood to be the point in time at which the contrast medium flows into the aorta of a patient. A method of determining the Care-Bolus time is known to the person skilled in the art. The image data assigned to the Care-Bolus time may have a high contrast, so that a segmentation may be advantageously carried out.

The at least one region of interest may be defined automatically and/or manually, (by an operator, for example), and/or semi-automatically, for example, by at least one region of interest, which may be adapted and/or confirmed by an operator, being proposed automatically by a processor unit. This makes possible a precise and convenient determination of the at least one region of interest.

One form of embodiment makes provision for the second reconstructed data to be established only for the at least one region of interest. Advantageously, this enables the region, in which a possible computing-intensive establishment of the second reconstructed data is to be carried out, to be specified and/or restricted. In addition, a restriction of the second reconstructed data enables its possible evaluation, (by a radiologist, for example), to be simplified, since less data, (e.g., images), is to be viewed and/or diagnosed in the evaluation.

It is further proposed that at least one temporal signal curve is established from the reconstructed data. The temporal signal curve may include a signal intensity, which is represented over a period of time, for example. The consideration of a temporal signal curve, (e.g., by ignoring spatial information), makes possible a simplified evaluation, for example, compared with an evaluation of a 4D dataset. The temporal signal curve enables a contrast medium curve to be shown. Through the thus known patient-specific contrast medium curve the timing for possibly one or more further reconstructions of the provided measured values, (e.g., the establishment of second reconstructed data), may be configured to a specific patient.

The at least one temporal signal curve may only take account of first reconstructed data from the at least one region of interest. Such a restriction of the data to be processed makes possible a more precise and more informative evaluation of the measurement data. It is conceivable for a temporal signal curve to be established for each defined region of interest.

Depending on the definition of the at least one region of interest, different contrast medium phases, (e.g., venous phase and/or arterial phase), may be shown. If the at least one region of interest includes a vein, for example, the associated temporal signal curve may include a venous contrast medium phase. The same naturally also applies for an artery.

One embodiment makes provision for at least one time window to be defined within the at least one temporal signal curve established. It is conceivable for one or more time windows to be established for each temporal signal curve. A time window may be defined, for example, by a start time and a duration or a start time and an end time.

The definition of the at least one time window makes a further optimization of the method possible. Through the definition of the at least one time window an individual or patient-specific generation of reconstruction parameters may be achieved. In particular, optimum account may be taken of a contrast medium course and/or a distribution of a contrast medium in an imaging volume.

The at least one time window may be defined automatically and/or manually by an operator and/or semi-automatically. An automatic definition, (with the aid of a processing unit, for example), enables the sequence of the contrast medium examination to be designed more efficiently. In a semi-automated variant, for example, a possible manual definition may be supported by an automated pre-evaluation.

Above and beyond this it is conceivable for further reconstruction parameters to be defined as well as the at least one time window, such as a temporal resolution. The definition of these types of further reconstruction parameters may also be done automatically and/or manually and/or semi-automatically.

It is further proposed that the at least one time window is defined by at least one peak value of the at least one temporal signal curve. This variant is advantageous since the at least one peak value may represent characteristic features of a temporal sequence, for example, in respect of a possible arterial and/or venous phase and/or late phase of a contrast medium distribution in a body. Methods for peak detection are known to the person skilled in the art. The at least one time window may be defined such that it includes at least one peak value, so that a possible start time of the time window lies before the at least one peak value and the end time of the time window lies after the at least one peak value.

A dynamic contrast medium examination may be subdivided into different examination phases. One of the at least one time windows may depend on an arterial phase and/or a venous phase and/or a late phase. These phases are revealing for evaluation or diagnosis, for example, by a radiologist.

Advantageously, the establishment of the second reconstructed data is only carried out in the at least one time window. Thus, for example, the only data, (e.g., image data), present for a possible evaluation of the second reconstructed data, is previously selected data, for example, entirely or in part by an operator. This means, for example, that a radiologist may be provided with as little data as possible and as much as is necessary for diagnosis.

The reliable optimal definition of the at least one time window enables the likelihood to be increased that any measured value recording or the tedious calculations for establishment of the second reconstructed data only have to be carried out once. The reconstruction having to be carried out repeatedly, possibly iteratively, in order to achieve a precise timing, may be prevented. That means a simplification for a work sequence of an operator, (for example, a medical-technical assistant (MTA)), for the recording of any measured values, since attention does not have to be paid to critical timing here. For example, it does not have to be insured that any reconstruction windows defined before the measurement also have to map the desired contrast medium phase correctly. Above and beyond this any renewed administration of contrast medium that might be needed may be avoided.

Furthermore, a medical imaging device is provided, wherein the medical imaging device is configured to carry out a method for evaluation of a dynamic contrast medium distribution.

The advantages of the medical imaging device correspond to the advantages of the method for evaluation of a dynamic contrast medium evaluation, which have been set down in detail above. Features, advantages or alternate forms of embodiment mentioned here may also likewise be transferred to the other claimed subject matter and vice versa. Thus it is made possible, by a generation of optimum patient-specific reconstruction parameters, to obtain reliably reusable reconstruction results. In particular, the determination of the reconstruction parameters may be simplified or supported in the best possible manner by the medical imaging device.

In addition, it is conceivable for the medical imaging device to include a contrast medium injector, through which a contrast medium may be administered to a possible patient.

Furthermore, a computer program product is proposed that includes a program and is able to be loaded directly into a memory of a programmable system control unit, wherein the computer program product is configured to carry out a method for evaluation of a dynamic contrast medium distribution when the program is executed in the system control unit of the medical imaging device.

### BRIEF DESCRIPTION OF THE DRAWINGS

FIG. 1 depicts a block diagram of an example of a method.

FIG. 2 depicts a block diagram explaining an act in one embodiment in greater detail.

FIG. 3 depicts an example of two temporal signal curves of first reconstructed data.

FIG. 4 depicts a basic diagram example of a magnetic resonance device.

### DETAILED DESCRIPTION

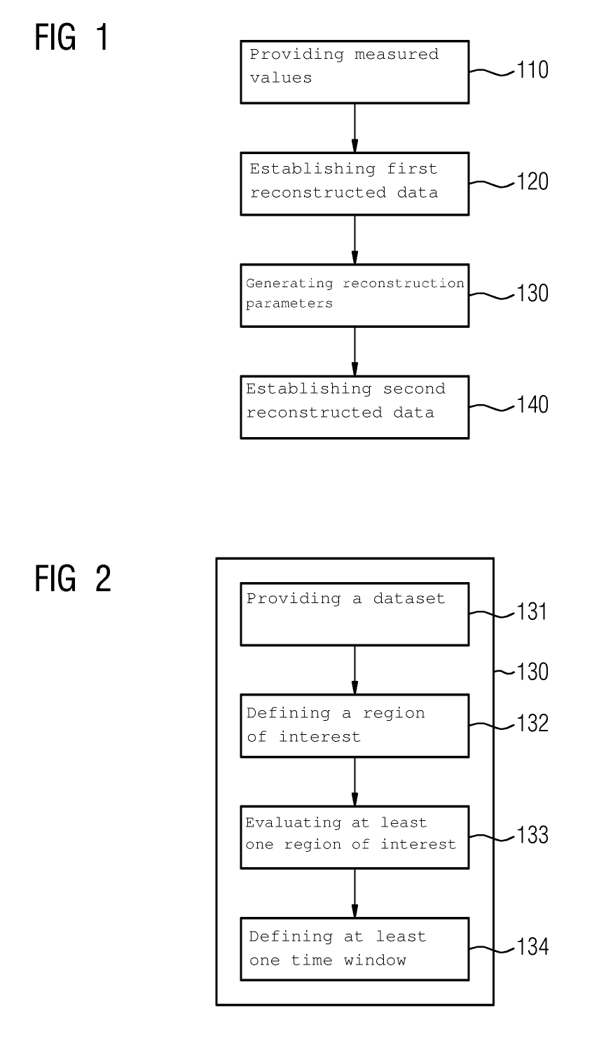

FIG. 1 illustrates an example of a method. In act 110, measured values of a medical imaging examination are provided. The measured values may include dynamic imaging information of an examination object, e.g., of a living being or patient. This provision is independent of a time at which the measured values were recorded. Thus, the provision of the measured values may include a process of loading into a main memory stored measured values that were detected at any given previous point in time, for example, by a medical imaging device, and were stored on a storage medium. The measured values may also be provided directly while a measurement is being carried out. Suitable measurement methods for this are, in particular, sparse matrix techniques, (e.g., the GRASP technique), since these methods may detect dynamic processes very quickly.

In act 120, first reconstructed data is established from a first subset of the measured values. The first subset may include all measured values provided, e.g., without restriction in respect of a time range of the dynamic measured values recorded. In order to achieve a short reconstruction time, the reconstruction 120 may be performed with a low temporal and/or spatial resolution, for example. The result of this reconstruction 120 may be a 4D dataset, e.g., a dataset including time-dependent, three-dimensional image data.

Acts 130 and 140 may be performed immediately after act 120. It is also conceivable for the establishment of the first reconstructed data to have already been undertaken as part of the measured value recording 120 at an earlier point in time and for the further evaluation in the acts 130 and 140 only to be carried out at a later point in time.

Reconstruction parameters are generated from the first reconstructed data in act 130, which are used in act 140 in order to establish second reconstructed data from a second subset of the measured values. This second reconstructed data may likewise represent a 4D dataset, which may be used by a radiologist for diagnosis, for example.

The act 130 is illustrated in greater detail in FIG. 2. For its part, act 130 may be subdivided into further acts. In act 131, a dataset, e.g., a 3D dataset and/or a volume dataset, is provided. This may be done by a point in time being selected from the 4D dataset reconstructed in act 120, e.g., the time that corresponds to the Care-Bolus time. The 3D dataset and/or volume dataset resulting therefrom may, for example, be displayed to an operator and/or be processed automatically.

In act 132, on the basis of this 3D dataset and/or volume dataset, at least one Region Of Interest (ROI) is defined. This definition may be done automatically and/or manually by an operator and/or semi-automatically. For example, a first region of interest may cover a vein and a second region of interest an artery within a body. It is conceivable for the second reconstructed data to be established in act 140 for just the at least one region of interest.

In act 133, the at least one region of interest is evaluated via the 4D dataset reconstructed in act 120 and a temporal signal curve is established for each of the at least one regions of interest. This at least one temporal signal curve may represent a maximum signal amplitude A over the time t. The maximum signal amplitude A at the respective time t may be the maximum amplitude value from all voxels located within the respective at least one region of interest.

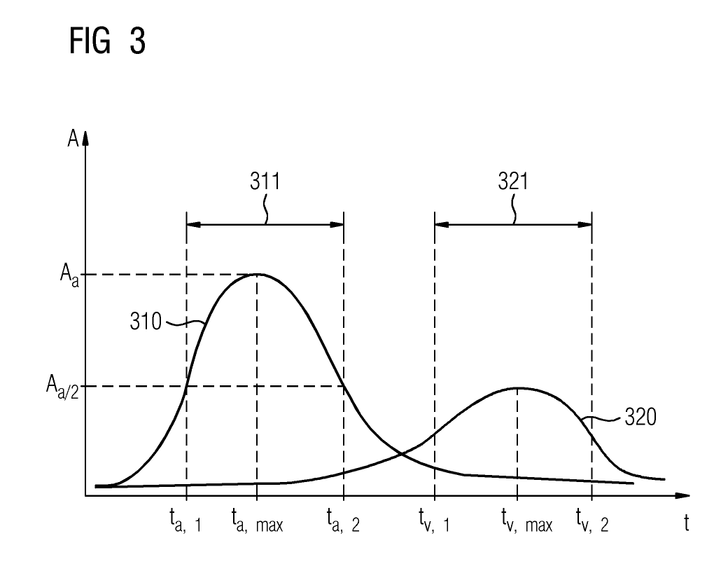

Depicted in FIG. 3, for example, are two such temporal signal curves 310 and 320. The curve 310 is derived from a region of interest, which includes an artery, so that 310 may be designated as an arterial curve. The curve 320 is derived from a region of interest, which includes a vein, so that 320 may be designated as a venous curve.

In act 134, at least one time window is defined within the at least one temporal signal curve, which may serve as reconstruction parameters for establishment of the second reconstructed data. In the example depicted in FIG. 3, these are the two time windows 311 and 321. The establishment of the second reconstructed data in act 140 may only be carried out with reference to the data that lies in this at least one time window 311, 321.

The proposed method guarantees through individual reconstruction parameters, configured to the respective measurement data, e.g., through the specific defined time window, that the second reconstructed data delivers good, reusable results. This enables timing problems to be avoided and multiple tedious reconstructions of the final image data otherwise often required may be avoided. Any doctor may receive only relevant images reconstructed, which is why the doctor has to view and diagnose fewer images than with conventional methods.

In the example depicted, one time window 311, 321 is defined for each temporal signal curve 310, 320. It is also conceivable for a number of time windows to be defined per temporal signal curve. The time window 311 relates to the arterial curve 310 and characterizes an arterial phase. The time window 321 relates to the venous curve 320 and characterizes a venous phase. A time window may be defined by a start time and an end time. In FIG. 3 the start time of the time window 311 is designated t_{a,1} and the end time of the time window 311 is designated t_{a,2}. The start time of the time window 321 is designated t_{v,1} and the end time of the time window 321 is designated t_{v,2}.

The at least one time window may be defined by at least one peak value of the at least one temporal signal curve. The peak value may be described by a maximum amplitude and/or a point in time at which the maximum amplitude occurs. In the case depicted, the amplitudes of the two curves are at a maximum at the times t_{a,max} and t_{v,max}. In addition, the maximum Aₐ is shown as exemplary for the arterial curve 320. The time window 311 may be defined, for example, by the times at which the curve 320 assumes a defined part of the maximum Aₐ. In the example depicted, the amplitude at the times t_{a,1} and t_{a,2} is equal to A_{a/2}=Aₐ/2. Another possibility for definition of the at least one time window consists of the start time t_{a,1} of the time window being defined by t_{a,1}=t_{a,max}−Δt_{a,1}, wherein Δt_{a,1} represents a defined time interval before the time of the maximum amplitude. Accordingly, the end time t_{a,1} may be defined by t_{a,2}=t_{a,max}+Δt_{a,2}, wherein Δt_{a,2} represents a defined time interval at the time of the maximum amplitude. Δt_{a,1} and Δt_{a,2} may be identical or they may differ. The described methods are of course also applicable for any other temporal signal curve, such as the venous curve 321. Other methods for defining the at least one time window 311, 321 are conceivable.

The at least one time window may be defined automatically and/or manually by an operator and/or semi-automatically. With a part-automated definition, a time window that may be changed manually by any operator may initially be proposed automatically.

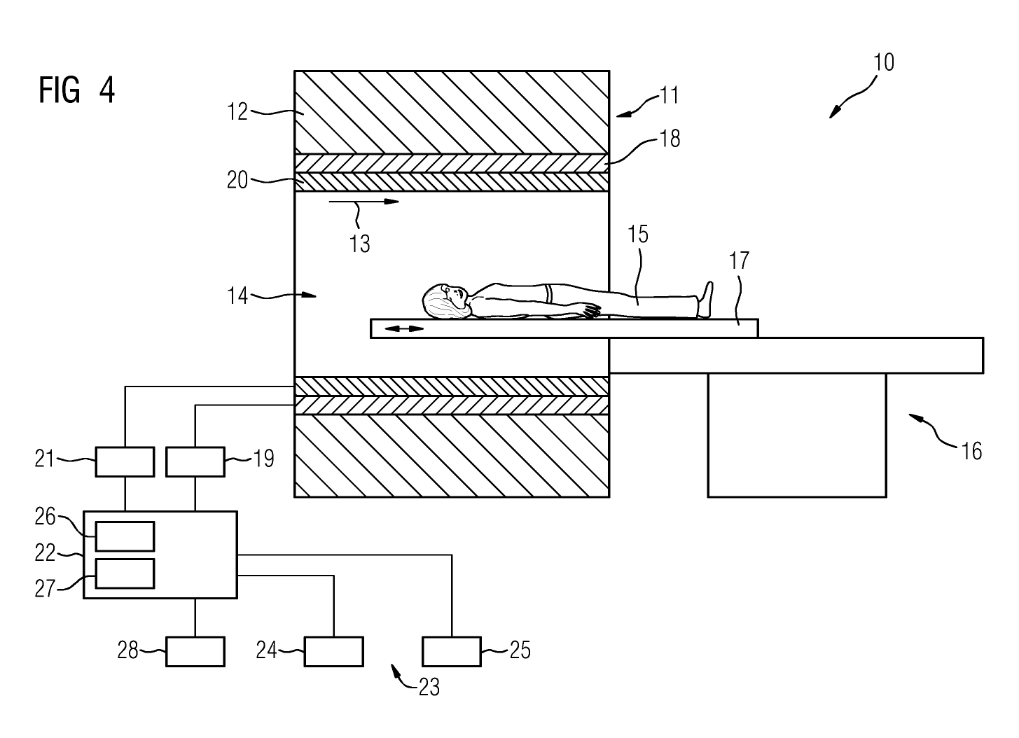

A magnetic resonance device 10 is depicted schematically in FIG. 4, which is configured to carry out the method described herein. The magnetic resonance device 10 includes a magnetic unit 11, which includes a superconducting main magnet 12 for creating a strong and temporally constant main magnetic field 13. In addition, the magnetic resonance device 10 has a patient receiving area 14 for receiving a patient 15. The patient receiving area 14 in the present exemplary embodiment is cylindrical in shape and is surrounded in a circumferential direction by the magnet unit 11. In other words, an embodiment of the patient receiving area 14 deviating therefrom is also conceivable. The patient 15 may be pushed by a patient support device 16 of the magnetic resonance device 10 into the patient receiving area 14. To this end, the patient support device 16 has a patient table 17 embodied movably within the patient receiving area 14.

The magnet unit 11 also has a gradient coil unit 18 for creating magnet field gradients, which are used for local encoding during imaging. The gradient coil unit 18 is controlled by a gradient coil control unit 19 of the magnetic resonance device 10. The magnet unit 11 further includes a radio-frequency antenna unit 20, which in the present exemplary embodiment is embodied as a body coil permanently integrated into the magnetic resonance device 10. The radio-frequency antenna unit 20 is designed for excitation of atomic nuclei, which occurs in the main magnetic field 13 created by the main magnet 12. The radio-frequency antenna unit 20 is controlled by a radio-frequency antenna control unit 21 of the magnetic resonance device 10 and radiates radio-frequency magnetic resonance sequences into an examination space, which is formed by a patient receiving area 14 of the magnetic resonance device 10. The radio-frequency antenna unit 20 is also embodied for receiving magnetic resonance signals.

For control of the main magnet 12, the gradient control unit 19 and for control of the radio-frequency antenna unit 21 the magnetic resonance device 10 has a system control unit 22. The system control unit 22 controls the magnetic resonance device 10 centrally, e.g., the carrying out of a pre-specified imaging gradient echo sequence. The system control unit 22 supports the execution of the method described above. To this end, it has a memory unit 26 and a processor unit 27, by which software and/or computer programs stored in the memory unit 26 are executed. In particular, a computer program as claimed in claim 18 may be executed with said unit.

Furthermore, the magnetic resonance device 10 includes a user interface 23, which is connected to the system control unit 22. Depending on embodiment, with the aid of the user interface 23 the at least one region of interest and/or the at least one time window 311, 321 may be defined with the at least temporal signal curve 310, 320 established. In addition, control information, (e.g., imaging parameters, as well as reconstructed magnet resonance images), may be shown on a display unit 24, (e.g., on at least one monitor), of the user interface 23 for a medical operator. Furthermore, the user interface 23 has an input unit 25, by which the information and/or parameters may be entered during and/or after a measurement process by the medical operating personnel.

The definition of the reconstruction parameters may be simply designed and supported in the best possible manner. As mentioned above, it is proposed that, in particular, a manual definition and/or modification of the at least one time window 311, 321 may be done in an operator-friendly manner via the user interface 23. Through simple adaptation of at least one graphical object shown on the display unit 24, (e.g., a box and/or a rectangle), such as by dragging and moving it in the way known from graphics programs, the time window may be modified in its position and duration by the input unit 25. In this case, the signal curve is advantageously simultaneously displayed over time by the display unit 24, such as within the at least one defined region of interest. Further reconstruction parameters, (e.g., a number of frames to be reconstructed and/or a number of datasets per phase and/or a temporal and/or spatial resolution), may possibly be modified by a context-sensitive menu with the aid of the input unit 25. Furthermore, it is conceivable that further graphical objects, such as boxes and/or rectangles, may be inserted and/or deleted via the user interface 23, in order to reconstruct additional phases. It is further conceivable for at least one preset default configuration to be able to be stored, for example, a configuration consisting of three phases, defined by three time windows, each of 15 seconds duration and an interval of 30 seconds, as well as a further configuration with two phases, defined by two time windows of 50 or 120 seconds duration and an interval of 10 seconds. This at least one preset configuration stored may also be adapted for individual or specific patients.

In one embodiment, the magnetic resonance device 10 also has a contrast medium injector 28. The contrast medium injector 28 may be used for administering a contrast medium to the patient 15. The contrast medium injector 28, (e.g., the timing of the injector), may be controlled by the system control unit 22.

The magnetic resonance device 10 shown in the present exemplary embodiment may include further components that magnetic resonance devices normally feature. The person skilled in the art knows how a magnetic resonance device 10 functions, so a more detailed description of the components is not provided here.

The enclosed drawings, the technical content, and the more detailed description relate to certain embodiments, which however are not to be regarded as a restriction of the inventive subject matter.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for evaluation of a dynamic contrast medium examination, the method comprising:
providing measured values;
establishing a first reconstructed data from a first subset of the measured values;
generating reconstruction parameters from the first reconstructed data; and
establishing a second reconstructed data from a second subset of the measured values, using the generated reconstruction parameters.

2. The method of claim 1, wherein the measured values comprise imaging information of an examination object.

3. The method of claim 1, wherein the measured values are detected by a medical imaging device.

4. The method of claim 3, wherein the detected measured values are detected using a sparse matrix technique.

5. The method of claim 1, wherein the first reconstructed data has a lower resolution than the second reconstructed data.

6. The method of claim 1, wherein at least one region of interest is defined for generation of the reconstruction parameters.

7. The method of claim 6, wherein the at least one region of interest is defined automatically, semi-automatically, manually by an operator, or a combination thereof.

8. The method of claim 7, wherein the second reconstructed data is only established for the at least one region of interest.

9. The method of claim 6, wherein the second reconstructed data is only established for the at least one region of interest.

10. The method of claim 1, wherein at least one temporal signal curve is established from the reconstructed data.

11. The method of claim 10, wherein at least one region of interest is defined for generation of the reconstruction parameters, and wherein the at least one temporal signal curve only takes account of first reconstructed data from the at least one region of interest.

12. The method of claim 10, wherein at least one time window is defined within the at least one temporal signal curve established.

13. The method of claim 12, wherein the at least one time window is defined automatically, semi-automatically, manually by an operator, or a combination thereof.

14. The method of claim 13, wherein the at least one time window is defined by at least one peak value of the at least one temporal signal curve.

15. The method of claim 12, wherein one time window of the at least one time window depends on an arterial phase, a venous phase, a late phase, or a combination thereof.

16. The method of claim 12, wherein the establishment of the second reconstructed data is only carried out in the at least one time window.

17. A medical imaging device comprising:
a system control unit having a memory unit and a processor unit,
wherein the medical imaging device detects measured values of a patient being examined, and
wherein the medical imaging device, with the system control unit, is configured to:
establish a first reconstructed data from a first subset of the measured values;
generate reconstruction parameters from the first reconstructed data; and
establish a second reconstructed data from a second subset of the measured values, using the generated reconstruction parameters.

18. The medical imaging device of claim 17, further comprising a contrast medium injector for administering a contrast medium to the patient.

19. A computer program product comprising a program, the computer program product being configured to be loaded directly into a memory of a programmable system control unit of a medical imaging device, wherein, when the program is executed in the programmable system control unit of the medical imaging device, the medical imaging device is configured to at least perform:
establish a first reconstructed data from a first subset of measured values;
generate reconstruction parameters from the first reconstructed data; and
establish a second reconstructed data from a second subset of the measured values, using the generated reconstruction parameters.
